(19) United States
(12) Patent Application Publication
(10) Pub. No.: US 2007/0116756 A1
(43) Pub. Date: May 24, 2007

(54) STABLE PHARMACEUTICAL COMPOSITIONS

(75) Inventors: Prakash Reddy Komireddi, Karimnagar (IN); Gurvinder Singh, New Delhi (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: Dr. Reddy's Laboratories Limited, Hyderabad (IN); Dr. Reddy's Laboratories, Inc., Bridgewater, NJ (US)

(21) Appl. No.: 11/561,624

(22) Filed: Nov. 20, 2006

Related U.S. Application Data
(60) Provisional application No. 60/746,322, filed on May 3, 2006.

(30) Foreign Application Priority Data
Nov. 23, 2005 (IN) 1716/CHE/2005

(51) Int. Cl. A61K 31/60; A61K 31/549; A61K 31/401; A61K 31/366; A61K 31/22; A61K 9/24; A61K 9/48

(52) U.S. Cl. 424/451; 424/472; 514/165; 514/423; 514/460; 514/548; 514/223.5; 514/651

(57) ABSTRACT

A pharmaceutical dosage form, comprising an outer capsule containing at least one capsule, tablet, and/or particles comprising different drug substances.

### INTRODUCTION TO THE INVENTION

The present invention relates to stable pharmaceutical compositions of cardiovascular therapeutic agents comprising a combination of therapeutically effective doses of a cholesterol-lowering agent, an inhibitor of the renin-angiotensin system, a diuretic, aspirin and optionally at least one beta-adrenergic receptor blocking agent, or their pharmaceutically acceptable salts, solvates, enantiomers or mixtures thereof in a single dosage form, the processes for preparing the same and methods of use and treatment for patients with cardiovascular diseases (“CVD”).

An aspect of the present invention also relates to stable pharmaceutical compositions comprising an outer capsule, which further comprises at least a smaller inner capsule or a smaller tablet (mini-tablet).

3-hydroxy-3-methylglutaryl-coenzyme A (“HMG-CoA”) reductase catalyzes the conversion of HMG-COA to mevalonate, which is an early and rate-limiting step in the biosynthesis of cholesterol. Inhibitors of HMG Co-A reductase are useful as cholesterol lowering agents, and are frequently referred to as “statin drugs.” Useful cholesterol-lowering agents include but are not limited to HMG CoA reductase inhibitors, bile acid sequestrants, probucol, fibric acid agents and intestinal cholesterol absorption inhibitors like ezetimibe. HMG-COA reductase inhibitors are among the useful cholesterol reducing agents for the present invention. HMG-COA reductase inhibitors that may be used in the present invention include, but are not limited to atorvastatin, pravastatin, cerivistatin, fluindostatin, fluvastatin, lovastatin, mevastatin, rosuvastatin, pitvastatin, dalvastatin and velostatin.

Pravastatin (Formula I), chemically is 1-Naphthalene-heptanoic acid, 1,2,6,7,8,8a-hexahydro-β,δ,6-trihydroxy-2-methyl-8-(2-methyl-1-oxobutoxy)-[1S-[1α(βS*,δS*),2α,6α,8β(R*),8aα]. It is commercially available as the sodium salt in the form of oral tablets of 10, 20, 40 and 80 mg strengths under the brand name PRAVACHOL™ and is manufactured by Bristol-Myers Squibb.

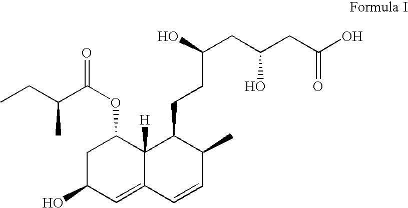

Low doses of aspirin (Formula II) affect platelet function by primarily inhibiting platelet cyclooxygenase (“COX”) thereby preventing the formation of the aggregating agent thromboxane A2. This action is irreversible and the effects last for the lifetime of the platelet. Anti-platelet aggregating agents like aspirin, analgrelide, dipyridamole, clopidogrel and ticlopidine are useful in the present invention. Aspirin chemically is acetylsalicylic acid.

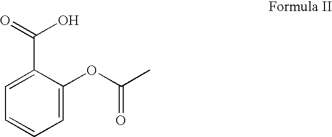

Inhibition of the renin-angiotensin system by the angiotensin converting enzyme (“ACE”) results in decreased plasma levels of angiotensin II, which leads to decreased vasopressor activity and aldosterone secretion. Inhibitors of the renin-angiotensin system are classified as ACE inhibitors and angiotensin 11 receptor antagonists (“ARBs”). ACE inhibitors that are useful in the present invention include, but are not limited to, captopril, cilazapril, delapril, enalapril, fentiapril, fosinopril, indolapril, lisinopril, perindopril, pivopril, quinapril, ramipril, spirapril, trandolapril, and zofenopril.

Lisinopril (Formula III), chemically is 1-{N²-[(S)-1-Carboxy-3-phenylpropyl]-L-lysyl}-L-proline dihydrate. It is commercially available in the form of oral conventional tablets of 2.5, 5, 10, 20 and 40 mg strengths under the brand name PRINIVIL™ and is manufactured by Merck.

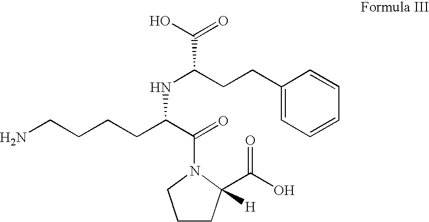

Thiazides and thiazide-like diuretics that are useful in the present invention include, but are not limited to, the drugs bendroflumethazide, chlorthiazide, chlorthalidone, hydrochlorthiazide, hydroflumethazide, indapamide, methyclothazide, metolazone, mefruside, polythiazide, trichlormethazide, cyclopenthiazide, polythiazide, qiunethazone and xipamide.

Hydrochlorothiazide (Formula IV), chemically is 6-chloro-3,4-dihydro-2H-1,2,4-benzothiadiazine-7-sulfonamide-1,1 -dioxide. It is a diuretic and antihypertensive agent. It acts on kidneys to reduce sodium reabsorption in the distal convoluted tubule. Hydrochlorothiazide is commercially available in the form of 25 mg and 50 mg oral tablets manufactured by Ciba Geneva, under the trade name ESIDRIX™.

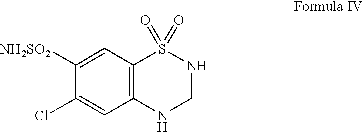

Useful cardiovascular drugs further include beta-adrenergic receptor antagonists that competitively inhibit binding of norepinephrine to its receptors, and are used in the treatment of essential hypertension. These drugs include atenolol, bisoprolol (typically used as the fumarate salt), labetolol (typically as the hydrochloride salt), metoprolol (typically as the hydrochloride salt), propranolol (typically as the hydrochloride salt), and several others. Other salts can be used for the salt-forming compounds.

In conventional therapy, patients at higher risk of CVD are frequently on multiple drug therapy, taking two or more different medications at the same time. Presenting multiple medications in a single composition promotes patient compliance by avoiding the inconvenience of taking multiple doses of medicines in a single day and reducing the chances of skipping doses.

Combining two or more active ingredients in single dosage form has critical considerations, due to the possibility of chemical interactions between the drug substances. Acidic active ingredients like aspirin can react with basic drugs and acidic ingredients such as aspirin can cause the degradation of acid labile drugs. Hence a composition comprising a combination of active ingredients, wherein the formation of impurities has been controlled within the ICH limits, is highly desirable.

International Application Publication No. WO 2005/011586 describes a pharmaceutical dosage form comprising therapeutic amounts of: a beta-adrenergic receptor antagonist, a diuretic or both; a cholesterol-lowering agent; an inhibitor of the renin-angiotensin system and aspirin wherein acidic components are separated from basic components.

U.S. Patent Application Publication No. 205/0053648 discloses a medication delivery device for simultaneous delivery of multiple drugs, wherein the multiple drugs are encased within separate containers.

U.S. Patent Application Publication No. 2003/0068366 describes an oral dosage form comprising a combination of therapeutically effective doses of a cholesterol lowering agent, a renin-angiotensin system inhibitor, and aspirin where two active agents are present in dosage units that separates the two from the other active agent and also from each other.

Great Britain Patent No. 2361185 describes a formulation and its method of preparation. The formulation comprises active principals from at least two categories chosen from at least one blood pressure lowering agent, at least one lipid regulating agent, at least one platelet function-altering agent, and/or at least one plasma/serum homocysteine-lowering agent.

The need to use cardiovascular therapeutic agents with different and complementary mechanisms of action frequently arises in the treatment of CVD due to the progressive nature of the disease with deterioration over time. The use of a single active is inadequate for this purpose

However, the selection of a suitable dosage form comprising combinations of actives depends on variables like the physicochemical properties of the active agents and excipients, the dose to be administered, and the intended site of action. Also a combination of active agents is difficult to formulate due to the inherent differences in physicochemical properties, drug-drug and drug-excipient incompatibilities, and the type of drug release profile needed to elicit the necessary therapeutic efficacy.

There are no known available fixed dose pharmaceutical compositions as disclosed in the present invention in the field of clinical practice. Thus the present invention fills a highly desirable gap in the medical art for the treatment of CVD by improving patient compliance due to the ease of administration, reduced frequency of dosing and reduction in the doses of the actives used in combination because of the synergistic action, thus resulting in a reduction of side effects.

This and other such needs are addressed by the instant invention.

### SUMMARY OF THE INVENTION

An aspect of the present invention provides for stable pharmaceutical compositions comprising therapeutic amounts of a cholesterol-lowering agent, an inhibitor of the renin-angiotensin system, a diuretic, aspirin and optionally at least one beta-adrenergic receptor blocking agent.

Another aspect of the present invention provides for processes for preparing stable pharmaceutical compositions comprising a cholesterol-lowering agent, an inhibitor of the renin-angiotensin system, a diuretic, aspirin and optionally at least one beta-adrenergic receptor blocking agent.

Still further aspect of the present invention provides for methods of using stable pharmaceutical compositions comprising a cholesterol-lowering agent, an inhibitor of the renin-angiotensin system, a diuretic, aspirin and optionally at least one beta-adrenergic receptor blocking agent for the treatment of patients with CVD.

In an embodiment, the stable pharmaceutical compositions of the present invention comprise an outer capsule, which further comprises at least a smaller inner capsule.

In another embodiment, the stable pharmaceutical compositions of the present invention comprise an outer capsule, which further comprises at least a smaller tablet.

An embodiment of the invention includes a pharmaceutical dosage form, comprising a capsule containing:
a) a capsule, tablet, or particles comprising one or both of aspirin and a cholesterol-lowering agent; and
b) a capsule, tablet, or particles comprising one or more of a cholesterol-lowering agent, a beta-adrenergic receptor blocking agent, a diuretic, and an inhibitor of the renin-angiotensin system;
   wherein only one of a) and b) is in the form of particles.

Another embodiment of the invention includes a pharmaceutical dosage form, comprising a capsule containing:
a) a capsule, tablet, or particles comprising aspirin; and
b) a capsule, tablet, or particles comprising one or more of a cholesterol-lowering agent, a beta-adrenergic receptor blocking agent, a diuretic, and an inhibitor of the renin-angiotensin system;
   wherein only one of a) and b) is in the form of particles.

### DETAILED DESCRIPTION OF THE INVENTION

The present invention relates to stable pharmaceutical compositions comprising therapeutic amounts of a cholesterol-lowering agent, an inhibitor of the renin-angiotensin system, a diuretic, aspirin and optionally at least one beta-adrenergic receptor blocking agent or their pharmaceutically acceptable salts, solvates, enantiomers or mixtures thereof in a single dosage form, processes for preparing the same, and methods of use and treatment for patients with CVD.

In context of the present invention, the combined administration of cardiovascular therapeutic agents results in a beneficial and potentiating/synergistic therapeutic effect. Moreover the therapeutic benefits resulting from combined treatment are prolongation of efficacy, a broad therapeutic treatment of diseases and conditions associated with CVD.

The invention also relates to a treatment for patients having an elevated risk of cardiovascular events such as myocardial infarction (heart attack), cardiac arrest, congestive heart failure, stroke, peripheral vascular disease, and claudication. The risk factors associated with such life-threatening events include genetic predisposition, tobacco smoking, diabetes, elevated serum cholesterol, hypertension, systemic lupus erythematosus, prior heart attacks or strokes, hemodialysis, elevated homocysteine levels, obesity, sedentary lifestyles, receiving an organ transplant, and others.

In an embodiment, the stable pharmaceutical compositions comprise an outer capsule, which further comprises at least a smaller inner capsule comprising cardiovascular therapeutic agents. The active ingredients can be present in one or more small capsules either alone or in combination with one or more other actives. Similarly the outer capsule may contain active(s) optionally in combination with one or more other actives.

In another embodiment, the stable pharmaceutical compositions comprise an outer capsule, which further comprises at least a smaller tablet comprising cardiovascular therapeutic agents. The active ingredients can be present in one or more small tablets either alone or in combination with one or more other actives. Similarly the outer capsule may contain active(s) optionally in combination with one or more other actives.

In an embodiment, one or more of the active ingredients will be present within an outer capsule in the form of particles, such as a powder and/or a granulated solid, while one or more other active ingredients will be present within the outer capsule in the form of one or more smaller capsules and/or tablets.

In one embodiment, the combination of pravastatin, lisinopril and hydrochlorothiazide with aspirin has been found to be useful within the context of the present invention.

In an embodiment, the present invention provides a fixed dose of pravastatin in the range of about 5-100 or about 10-80 mg/day, lisinopril in the range of about 2.5-80 or about 2.5-40 mg/day, hydrochlorothiazide in the range of about 10-100 or about 25-100 mg/day, and aspirin in the range of about 25-600 or about 50-160 mg/day.

In one of the embodiments of the present invention, the stable capsule compositions comprise aspirin filled in a smaller inner capsule, which in combination with a blend composition comprising pravastatin, lisinopril and hydrochlorothiazide, is placed an outer capsule.

In some embodiments of the present invention, the stable capsule compositions comprise a smaller tablet composition comprising pravastatin, lisinopril and hydrochlorothiazide, and a blend composition comprising aspirin, wherein the smaller tablet and aspirin blend are combined and filled in an outer capsule.

Other therapeutic agents that are useful in the present invention include, but are not limited to: diuretics like inhibitors of carbonic anhydrase, loop diuretics, potassium sparing diuretics, and antagonists of mineralocorticoid receptors; antidiabetics, including biguanides such as metformin (GLUCOPHAGE®) and phenformin (DBI®), sulfonylureas such as acetohexamide (DYMELOR®), chlorpropamide (DIABINASE®), glimepiride (AMARYL®), glipizide (GLUCOTROL®, GLUCOTROL XL®), glyburide (DIABETA®, GLYNASE®, MICRONASE®), tolazamide (TOLINASE®), tolbutamide (ORINASE®), glitazones such as rosiglitazone (AVANDIN®), troglitazone (PRELAY®, REXULIN®) and pioglitazone (ACTOS®); glucosidase inhibitors such as acarbose (PRECOSE®) and meglitol (GLYSET®); meglitinides such as nateglinide (STARLIX®) and repaglinide (NOVONORM®); lipid lowering agents such as fibrates including clofibrate (ATROMID-S®), gemfibrozil (LOPID®), fenofibrate (TRICOR®); and the like can be used in the invention. The active ingredients in the invention can be presented with or without a pharmaceutical aid.

Common diluents that can be used in pharmaceutical formulations include microcrystalline cellulose (“MCC”), silicified MCC (e.g. PROSOLV™ HD 90), microfine cellulose, lactose, starch, pregelatinized starch, sugar, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and the like.

Binders can be included in the pharmaceutical compositions of the present invention to help hold tablets together after compression. Some typical binders are acacia, guar gum, alginic acid, carbomer (e.g. carbopol), dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethylcellulose sodium, liquid glucose, magnesium aluminum silicate, polymethacrylates, povidone (e.g. Povidone K-90 D, KOLLIDON®, PLASDONE®), gelatin, and starch.

The pharmaceutical compositions to be made into tablets may further include a disintegrant. Disintegrants include, but are not limited to, methyl cellulose, microcrystalline cellulose, carboxymethyl cellulose calcium, carboxymethyl cellulose sodium (e.g. AC-DI-SOL®, PRIMELLOSE®), crospovidone (e.g. KOLLIDON®, POLYPLASDONE®), povidone K-30, guar gum, magnesium aluminum silicate, colloidal silicon dioxide (AEROSIL®), polacrilin potassium, starch, pregelatinized starch, sodium starch glycolate (e.g. EXPLOTAB®), and sodium alginate.

The compositions for tabletting may further include additional pharmaceutically acceptable excipients, including one or more of glidants, lubricants, surfactants such as sodium lauryl sulphate, and other commonly used excipients. This list, and the foregoing listings of representative specific excipients, is not intended to be exhaustive, as those skilled in the art will be aware of other substances that can be used.

Formulation of present invention may include antioxidants including, but not limited to, ascorbic acid and its esters, butylated hydroxy toluene (BHT), butylated hydroxy anisole (BHA), a-tocopherol, cystein, citric acid, propyl gallate, and sodium bisulfite.

The process for manufacturing the formulation of present invention is not limited to the processes described in the application and the formulation can be prepared by using any of the processes known to one skilled in the art. One, or more than one, active ingredient can be used along with or without directly compressible grade excipients or granulated together or separately by wet granulation or dry granulation with or without excipients. Further one or more than one active(s) can be granulated while the others may be used as such without granulation. In an embodiment, the pharmaceutical compositions of the present invention are manufactured as described below. The granules of active(s) are prepared by sifting the actives and excipients through the desired mesh size sieve and then are mixed using a rapid mixer granulator or planetary mixer or mass mixer or ribbon mixer or fluid bed processor or any other suitable device. The blend can be granulated, such as by adding a solution of a binder whether alcoholic or hydro-alcoholic or aqueous in a low or high shear mixer, fluidized bed granulator and the like or by dry granulation. The granulate can be dried using a tray drier or fluid bed drier or rotary cone vacuum drier and the like. The sizing of the granules can be done using an oscillating granulator or comminuting mill or any other conventional equipment equipped with a suitable screen. Alternatively, granules can be prepared by extrusion and spheronization, or roller compaction.

Also the manufacture of granules of actives can be made by mixing the directly compressible excipients or by roller compaction. The blend so obtained can be compressed using a suitable device, such as a station rotary machine to form slugs, which are passed through a mill or fluid energy mill or ball mill or colloid mill or roller mill or hammer mill and the like, equipped with a suitable screen to obtain the milled slugs of actives.

In another aspect of the invention, the smaller tablets (mini-tablets) can be made by compressing the granules using die-and-punch of various sizes and shapes, as desired. Optionally, the coating on the tablets can be applied by techniques known to one skilled in the art such as spray coating, dip coating, fluidized bed coating and the like.

In one of the embodiments of the present invention, a suitable solvent system such as alcoholic or hydroalcoholic or aqueous or organic may be used.

The following examples will further illustrate certain aspects of the invention in greater detail and are not intended to limit the scope of the invention.

### EXAMPLE 1

Preparation of a Fixed Dose Formulation.

a) Composition of Smaller Inner Capsule:

| | Quantity |
|---|---|
| Ingredients | (mg/capsule) |
| Lisinopril | 5 |
| Atenolol | 25 |
| Dibasic calcium phosphate anhydrous (A-Tab ®)$ | 87.5 |
| Microcrystalline cellulose (Avicel ® PH 112)$$ | 40 |
| Mannitol (Pearlitol ® SD 200)* | 25 |
| Spectracol Ponceau ® 4RLK 815633** | 0.5 |
| Polyvinylpyrrolidone (Povidone ® K-30)# | 4 |
| Zinc stearate | 2 |
| Sodium starch glycolate | 8 |
| Zinc stearate | 3 |
| Total weight | 200 |

$Innophos manufacturers A-Tab ®
$$FMC Biopolymer manufactures Avicel ® PH 112.
*Pearlitol ® SD 200 is manufactured by Roquette
**M/s Sencient, Mumbai manufactures Spectracol Ponceau ® 4RLK 815633
#BASF manufacturers Polyvinyl pyrrolidone (Povidone ® K-30)

Manufacturing Process:
1. Ingredient no. 1 to 8 were passed through ASTM # 40 mesh sieve and mixed uniformly.
2. The ingredients of step 1 were dry granulated by roll compaction process.
3. The granules thus obtained were lubricated using ingredient 9 and 10.
4. The lubricated granules were filled in the capsules.

b) Components of Outer Capsule:

| | Quantity |
|---|---|
| Ingredients | (mg/capsule) |
| Simvastatin (with butylated hydroxy anisole 0.01%) | 10 |
| Acetyl salicylic acid (Rhodine ® 3126) | 75 |
| Lactose monohydrate (Flowlac ® 100)# | 68.24 |
| Pregelatinized starch (Starch ® 1500)## | 3.75 |
| Ascorbic acid | 2.5 |
| Butylated hydroxy anisole | 0.01 |
| Citric acid anhydrous | 1.25 |
| Microcrystalline cellulose (Avicel ® PH 112) | 2.5 |
| Zinc stearate | 0.5 |
| Pregelatinized starch (Starch ® 1500) | 6.25 |
| Microcrystalline cellulose (Avicel ® PH 112) | 2.5 |
| Zinc stearate | 2.5 |
| Total weight | 175 |

#FlowLac ®100 is manufactured by Meggle Pharma. It is the spray-dried alpha-lactose monohydrate.
##Colorcon manufacturers Starch ® 1500 which is partially pregelatinized maize starch.

Manufacturing Process:
1. Ingredients 1 through 9 were passed through ASTM 40# mesh sieve and mixed uniformly.
2. The ingredients of step 1 were dry granulated by roll compaction process.
3. The granules thus obtained were lubricated by adding ingredients 10, 11 and 12.
4. The inner capsule comprising lisinopril and atenolol was placed inside the body of the outer capsule.
5. Lubricated granules of step 3 were filled into the outer capsule of step 4 and sealed.

### EXAMPLE 2

Preparation of Fixed Dose Formulation.

a) Components of Smaller Inner Capsule:

| | Quantity |
|---|---|
| Ingredients | (mg/capsule) |
| Lisinopril | 5 |
| Hydrochlorothiazide | 12.5 |
| Dibasic calcium phosphate anhydrous (A-Tab ®) | 88 |
| Microcrystalline cellulose (Avicel ® PH 112) | 52 |
| Mannitol (Pearlitol ® SD 200) | 25 |
| Spectracol Ponceau ® 4RLK 815633 | 0.5 |
| Polyvinyl pyrrolidone (Povidone ® K-30) | 4 |
| Zinc stearate | 2 |
| Sodium starch glycolate | 8 |
| Zinc stearate | 3 |
| Total weight | 200 |

Manufacturing Process:

Similar to Example 1, except that hydrochlorothiazide was used in place of atenolol.

b) Components of Outer Capsule:

| | Quantity |
|---|---|
| Ingredients | (mg/capsule) |
| Simvastatin (with butylated hydroxy anisole 0.01%) | 10 |
| Acetyl salicylic acid (Rhodine ® 3126) | 75 |
| Lactose monohydrate (Flowlac ® 100) | 68.24 |
| Pregelatinized starch (Starch ® 1500) | 3.75 |
| Ascorbic acid | 2.5 |
| Butylated hydroxy anisole | 0.01 |
| Citric acid anhydrous | 1.25 |
| Microcrystalline cellulose (Avicel ® PH 112) | 2.5 |
| Zinc stearate | 0.5 |
| Pregelatinized starch (Starch ® 1500) | 6.25 |
| Microcrystalline cellulose (Avicel ® PH 112) | 2.5 |
| Zinc stearate | 2.5 |
| Total weight | 175 |

Manufacturing process was similar to that described in Example 1.

### EXAMPLE 3

Preparation of Fixed Dose Formulation.

a) Components of Smaller Inner Capsule:

| | Quantity |
|---|---|
| Ingredients | (mg/capsule) |
| Aspirin | 75 |
| Lactose Monohydrate (Flow Lac 100) | 60 |
| Microcrystalline cellulose NF (Avicel ® PH 112) | 12.5 |
| Zinc stearate | 1 |
| Zinc stearate | 1.5 |
| Total weight | 150 |

Manufacturing Process:
1. Ingredient no. 1 to 4 were passed through ASTM 40# mesh sieve and mixed uniformly.
2. The ingredients of step 1 were dry granulated by roll compaction process.
3. The granules thus obtained were lubricated using ingredient 5.
4. The lubricated granules were filled in the capsule.

b) Components of Outer Capsule:

| | Quantity |
|---|---|
| Ingredients | (mg/capsule) |
| Pravastatin sodium | 40 |
| Lisinopril | 10 |
| Hydrochlorothiazide | 12.5 |
| Meglumine | 2 |
| Dibasic calcium phosphate anhydrous (A-Tab ®) | 88 |
| Microcrystalline cellulose (Avicel ® PH 112) | 56 |
| Mannitol (Pearlitol ® SD 200) | 25 |
| Spectracol Ponceau ® 4RLK 815633 | 0.5 |
| Polyvinyl pyrrolidone (Povidone ® K-30) | 4 |
| Sodium starch glycolate NF | 8 |
| Zinc stearate | 4 |
| Total weight | 250 |

Manufacturing Process:
1. Ingredient no 1 to 9 were passed through ASTM # 40 mesh sieve and mixed uniformly.
2. The ingredients of step 1 were dry granulated by roll compaction process.
3. The granules thus obtained were lubricated by adding ingredients 10 and 11.
4. The inner capsule comprising aspirin was placed in the body of the outer capsule.
5. Lubricated granules of step 3 were filled into the outer capsule of step 4 and sealed.

### EXAMPLE 4

Preparation of Fixed Dose Formulation.

a) Components of Smaller Inner Capsule:

Composition and manufacturing process was similar as described for smaller inner capsule (a) in Example 1.

b) Components of Outer Capsule:

| | Quantity |
|---|---|
| Ingredients | (mg/capsule) |
| Atorvastatin calcium | 40 |
| Lisinopril | 10 |
| Hydrochlorothiazide | 12 |
| Meglumine | 2 |
| Dibasic calcium phosphate anhydrous (A-Tab ®) | 88 |
| Microcrystalline cellulose (Avicel ® PH 112) | 56 |
| Mannitol (Pearlitol ® SD 200) | 25 |
| Spectracol Ponceau ® 4RLK 815633 | 0.5 |
| Polyvinyl pyrrolidone (Povidone ® K-30) | 4 |
| Sodium starch glycolate | 8 |
| Zinc stearate | 4 |
| Total weight | 250 |

Manufacturing Process:

Similar to as described in Example 3, except that atorvastatin calcium was used in place of pravastatin sodium.

### EXAMPLE 5

Stability Testing of the Composition of Example 1.

The composition was directly exposed in an open petri dish to 40° C. and 75% relative humidity for 4 weeks, and then analyzed to evaluate the level of impurity formation.

| | | Percent |
|---|---|---|
| Ingredient | Impurity | by weight |
| Lisinopril | Monoacetyl impurity | Not detected* |
| Atenolol | Diacetyl impurity | 0.03 |
| Aspirin | Salicylic acid | 0.376 |

*Limit of detection for monoacetyl impurity for lisinopiril is 0.05 percent by weight using HPLC method.

As per the International Conference on Harmonization (ICH) guidelines for low dose drugs, the individual known impurities should not exceed more than 0.5% during the course of stability. The levels of impurities in this formulation are within the acceptable levels.

### EXAMPLE 6

Preparation of a Fixed Dose Formulation Based on Capsule-in-capsule Concept.

a) Components of Smaller Inner Capsule:

| | Quantity |
|---|---|
| Ingredients | (mg/capsule) |
| Aspirin | 75 |
| Lactose Monohydrate (Flow Lac 100) | 60 |
| Microcrystalline cellulose NF (Avicel ® PH 112) | 14 |
| Zinc stearate | 1 |
| Total weight | 150 |

Manufacturing Process was Similar to that Described in Part (a) of Example 3.

b) Components of Outer Capsule:

| | Quantity |
|---|---|
| Ingredients | (mg/capsule) |
| Pravastatin sodium | 40 |
| Lisinopril | 10 |
| Hydrochlorothiazide | 12.5 |
| Meglumine | 2 |
| Microcrystalline cellulose (Avicel ® PH 112) | 127 |
| Mannitol (Pearlitol ® SD 200) | 25 |
| Crospovidone | 15 |
| Polyvinyl pyrrolidone (Povidone ® K-30) | 4 |
| Croscarmellose sodium | 10 |
| Zinc stearate | 4 |
| Total weight | 250 |
| Grand total weight | 400 |

Manufacturing process was similar to that described in part (b) of Example 3.

### EXAMPLE 7

Preparation of a Fixed Dose Formulation Based on Tablet-in-capsule Concept.

a) Composition of Aspirin Blend to Be Filled in Outer Capsule:

| | Quantity |
|---|---|
| Ingredients | (mg/capsule) |
| Aspirin | 75 |
| Lactose Monohydrate (Flow Lac 100) | 60 |
| Microcrystalline cellulose NF (Avicel ® PH 112) | 14 |
| Zinc stearate | 1 |
| Total weight | 150 |

Manufacturing Process:
1. All the ingredients were passed through ASTM 40# mesh sieve and mixed uniformly to obtain a blend.

b) Composition of film coated smaller tablet to be filled in outer capsule:

| | Quantity |
|---|---|
| Ingredients | (mg/capsule) |
| Pravastatin sodium | 40 |
| Lisinopril | 10 |
| Hydrochlorothiazide | 12.5 |
| Meglumine | 2 |
| Microcrystalline cellulose (Avicel ® PH 112) | 127 |
| Mannitol (Pearlitol ® SD 200) | 25 |
| Crospovidone | 15 |
| Polyvinyl pyrrolidone (Povidone ® K-30) | 4 |
| Croscarmellose sodium | 10 |
| Zinc stearate | 4 |
| Core tablet weight | 250 |

FILM COATING

| | |
|---|---|
| Opadry AMB Translucent | 8.75 |
| Isopropyl alcohol* | 50.5 |
| Water* | 50.5 |
| Coated tablet weight | 258.75 |
| Grand total weight | 408.75 |

*Evaporated during coating process.

Manufacturing Process:
1. The first eight ingredients were passed through ASTM # 30 mesh sieve and mixed uniformly.
2. The ingredients of step 1 were dry granulated by roll compaction process.
3. The granules obtained in step 2 were lubricated by adding croscarmellose sodium and zinc stearate.
4. Blend of step 3 was compressed into tablets using suitable punches.
5. Opadry was dispersed in isopropyl alcohol and water to get uniform dispersion
6. Tablets of step 4 were coated dispersion of step 5.
7. Coated tablets were placed in capsule and filled with aspirin blend of part (a).

1. A pharmaceutical dosage form, comprising a capsule containing:
a) a capsule, tablet, or particles comprising one or both of aspirin and a cholesterol-lowering agent; and
b) a capsule, tablet, or particles comprising one or more of a cholesterol-lowering agent, a beta-adrenergic receptor blocking agent, a diuretic, and an inhibitor of the renin-angiotensin system;
wherein only one of a) and b) is in the form of particles.

2. The pharmaceutical dosage form of claim 1, wherein a tablet is coated with a polymer.

3. The pharmaceutical dosage form of claim 1, wherein a) does not comprise a cholesterol-lowering agent and b) comprises a cholesterol-lowering agent.

4. The pharmaceutical dosage form of claim 1, wherein a) is in the form of particles.

5. The pharmaceutical dosage form of claim 1, wherein a) is in the form of a capsule.

6. The pharmaceutical dosage form of claim 1, wherein a cholesterol-lowering agent comprises at least one of atorvastatin, pravastatin, cerivistatin, fluindostatin, fluvastatin, lovastatin, mevastatin, rosuvastatin, pitvastatin, dalvastatin and velostatin.

7. The pharmaceutical dosage form of claim 1, wherein a diuretic comprises at least one of bendroflumethazide, chlorthiazide, chlorthalidone, hydrochlorothiazide, hydroflumethazide, indapamide, methyclothazide, metolazone, mefruside, polythiazide, trichlormethazide, cyclopenthiazide, polythiazide, qiunethazone and xipamide.

8. The pharmaceutical dosage form of claims 1, wherein an inhibitor of the renin-angiotensin system comprises at least one of captopril, cilazapril, delapril, enalapril, fentiapril, fosinopril, indolapril, lisinopril, perindopril, pivopril, quinapril, ramipril, spirapril, trandolapril, and zofenopril.

9. The pharmaceutical dosage form of claim 1, wherein a beta-adrenergic receptor blocking agent comprises one or more of atenolol, bisoprolol or a salt thereof, labetolol or a salt thereof, metoprolol or a salt thereof, or propranolol or a salt thereof.

10. The pharmaceutical dosage form of claim 1, wherein a) does not comprise a cholesterol-lowering agent and b) comprises pravastatin, lisinopril, and hydrochlorothiazide.

11. A pharmaceutical dosage form, comprising a capsule containing:
a) a capsule, tablet, or particles comprising aspirin; and
b) a capsule, tablet, or particles comprising one or more of a cholesterol-lowering agent, a beta-adrenergic receptor blocking agent, a diuretic, and an inhibitor of the renin-angiotensin system;
wherein only one of a) and b) is in the form of particles.

12. The pharmaceutical dosage form of claim 11, wherein a tablet is coated with a polymer.

13. The pharmaceutical dosage form of claim 11, wherein a) is in the form of particles.

14. The pharmaceutical dosage form of claim 11, wherein a) is in the form of a capsule.

15. The pharmaceutical dosage form of claim 11, wherein a cholesterol-lowering agent comprises at least one statin drug.

16. The pharmaceutical dosage form of claim 11, wherein b) comprises pravastatin, lisinopril, and hydrochlorothiazide.

17. The pharmaceutical dosage form of claim 11, wherein a) is in the form of particles and b) comprises pravastatin, lisinopril, and hydrochlorothiazide.
